(19) United States
(12) Patent Application Publication
(10) Pub. No.: US 2004/0171082 A1
(43) Pub. Date: Sep. 2, 2004

(54) Diagnostic method

(76) Inventor: Martin Jeffrey, Penicuik (GB)

Correspondence Address: NIXON & VANDERHYE, PC, 1100 N GLEBE ROAD, 8TH FLOOR, ARLINGTON, VA, 22201-4714, US

(21) Appl. No.: 10/479,218

(22) PCT Filed: May 30, 2002
(86) PCT No.: PCT/GB02/02527

(30) Foreign Application Priority Data
May 31, 2001 (GB) 0113156.4

(51) Int. Cl. G01N 33/53; G01N 33/567

(52) U.S. Cl. 435/7.2

(57) ABSTRACT

A method for typing a strain of a transmissible spongiform encephalopathy (TSE) in an infected animal, said method comprising detecting the presence or concentration of a peptide sequence within a cell of a particular type taken from said animal, wherein the presence or concentration of said peptide sequence within said cell type is characteristic of a particular strain of TSE.

The present invention relates to a method of typing strains or forms of transmissible spongiform encephalopathies or prion disease found in infected animals, as well as to diagnostic kits and reagents used in the method.

The transmissible spongiform encephalopathies (TSEs) comprise a group of progressive neurological disorders characterised by neuroparenchymal vacuolation and accumulation of a disease specific isoform of a host coded cell surface sialoglycoprotein called prion protein (PrP). Scrapie, bovine spongiform encephalopathy (BSE) and variant Creutzfeldt-Jakob disease belong to this group of disorders. The diseases appear in various forms or strains.

Already, numerous TSE isolates (usually referred to as strains) have been identified following transmission of a range of sources into rodents. The possibility that some sheep may be infected with the BSE agent is of human and animal health concern.

Currently the only reliable method of TSE agent strain typing is based on the biological properties of an isolate following serial transmission in mice (Bruce et al., 1994, Transmission of bovine spongiform encephalopathy and scrapie to mice: strain variation and the species barrier. Philosophical Transactions-Royal Society of London. Series B. 343, 405-411). However, such methods are extremely time consuming and not all scrapie strains are readily transmitted to mice. Several studies have shown that the patterns of PrP deposition in the brains of mice with scrapie are strain specific (Bruce M E et al. 1989,. Neuroscience Letters 102, 1-6; Bruce 1996, Strain typing studies of scrapie and BSE. In Prion Diseases ed. Baker, H. F, and Ridley, R. M. pp. Totowa/NJ 07512: Humana Press Inc).

Several other methods have been studied in the hope of providing a more rapid answer to strain identification. All of these are based on properties of disease specific protease resistant fragments of PrP (PrP^{res}) such as the molecular weight (Parchi et al., 1996, Annals of Neurology 39, 767-778), ratio of glycoforms of the PrP^{res} fragments (Collinge et al., 1996, Nature 383, 685-690; Kuczius et al., 1998, Journal of Infectious diseases 178, 693-699; Somerville et al., 1997a, Nature 386, 564-564) or relative protease resistance of PrP^{res} (Kuczius and Groschup, 1999. Molecular Medicine 5, 406-418). However, these properties of PrP^{res} overlap when different strains or isolates are compared and so they cannot yet be used for definitive strain typing. A conformation assay of PrP^{res} has been described and may provide a means of strain typing but the usefulness of this technique has not yet been established (Safar et al., 1998, Nature Medicine 4, 1157-1165).

In brains from sheep with natural scrapie a number of different patterns of disease-specific PrP accumulation may be seen following immunohistochemical labelling (Ryder et al., 2001,. Veterinary Record 148, 7-13; van Keulen et al., 1995, Veterinary Pathology 32, 299-308).

The applicants have found that these patterns are associated with the accumulation and the inferred release of PrP by a number of different nervous system cell types including endothelial cells, astrocytes, neurons and ependymal cells and appear to be mainly influenced by differences in scrapie source (see Example 2 hereinafter and Gonzalez L. et al., Isolate and genotype effects on PrP accumulation in the brain of sheep naturally and experimentally affected with scrapie, J. Comparative Pathology, 2001 submitted). In the lymphoreticular system (LRS) tissues of both mice and sheep disease-specific PrP is recognised within tingible body macrophages (TBM) and around follicular dendritic cells (FDC) (Jeffrey et al., 2000, Journal of Pathology. 191, 323-332; Jeffrey et al., 2001b, Journal of Comparative Pathology In Press) of secondary follicles.

The applicants have found that immunohistochemistry, targeted to identify PrP peptide accumulation associated with particular cell types, can be used to distinguish strains of TSE, for example natural scrapie and BSE in sheep.

Furthermore, the applicants have found that the cellular and neuroanatomical distribution of disease specific PrP peptide fragments is different when BSE affected sheep and natural scrapie sources are compared. These differences appear to be related to the inferred in situ conformation dependent truncation pattern of disease specific PrP. In any event, the results suggest that the distribution of disease specific PrP epitopes in brain and LRS can allow strain typing of individual TSE disease, in particular sheep TSE disease.

According to the present invention there is provided a method for typing a strain of a transmissible spongiform encephalopathy (TSE) in an infected animal, said method comprising detecting the presence or concentration of a peptide sequence within a cell of a particular type taken from said animal, wherein the presence or concentration of said peptide sequence within said cell type is characteristic of a particular strain of TSE.

As used herein, the term “peptide sequence” refers to sequences, which are in the form of discrete peptides in isolation, or as part of a protein or truncated protein.

The particular cell type used in any particular case will vary depending upon factors such as the particular animal, the particular strains of TSE which infect it and the processing of the prion protein by the cells of the animal. In any particular case, this can be determined using methodology similar to that described hereinafter. Generally however, the cell type will be a particular brain cell type, a central nervous system (CNS) cell type, or a cell type contained within the LRS.

As demonstrated herein, immunohistochemistry has been used to identify specific prion protein (PrP) peptide sequences in specific cell types of the CNS and LPS of natural scrapie infected and BSE-agent-infected Suffolk and Romney sheep. These results indicate that immunohistochemical PrP profiling in the CNS and LRS of sheep allows the identification of sheep scrapie strains. These methods potentially allow recognition of TSE agent strain dependent differences in LRS and CNS pathogenesis and identification of multiple strains within an individual brain.

The differences which are noted amongst cell types may be due to isolate associated, cell specific in situ conformation dependant truncation pattern of disease specific PrP.

Preferably the method of the invention is used to detect the presence of a peptide sequence within a particular cell type, wherein the mere presence is characteristic of a particular strain of TSE. This allows the strain to be identified in an absolute manner.

In a particularly preferred embodiment, the method of the invention is applied to sheep in order to distinguish scrapie from BSE strains of TSE.

The results reported here show that antibody 521.7 generated from the peptide sequence 84-105 is not readily detected within presumed lysosomes of BSE agent infected CNS and LRS phagocytic cells but that the same peptide sequence is present in the same cells of natural scrapie affected sheep. Clinically affected and some pre-clinical BSE agent infected sheep could be differentiated from scrapie by the lesser amount of labelling of PrP containing the ovine 84-105 amino-acid peptide sequences in phagocytic cells of the LRS and brain.

Thus, a particularly preferred embodiment of the invention relies on the detection of the presence of a peptide sequence which binds to an antibody raised to a peptide corresponding to amino acids 84-105 of the prion protein of ovine spongiform encephalopathy or an epitopic region thereof, in a glial cell of an infected animal.

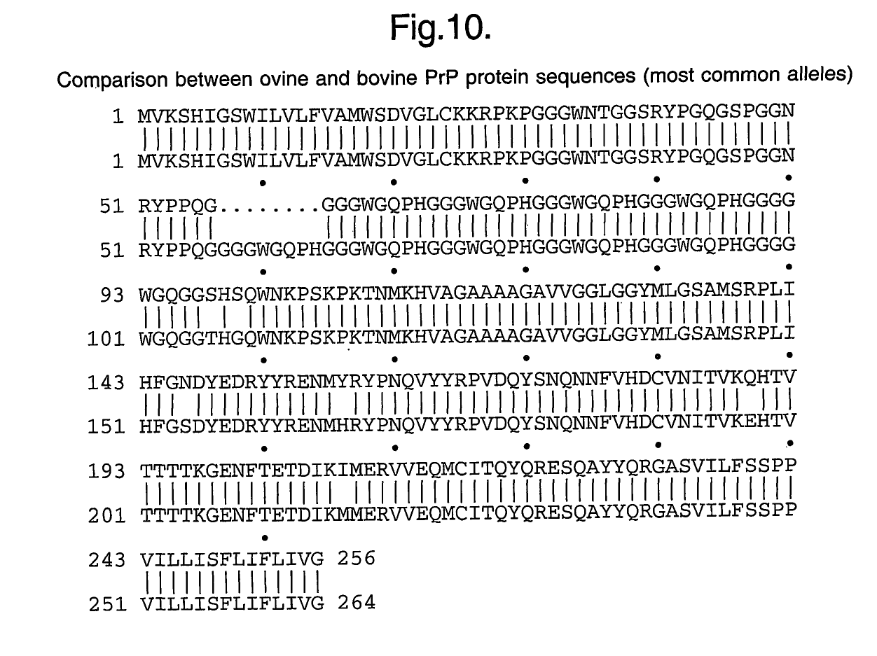

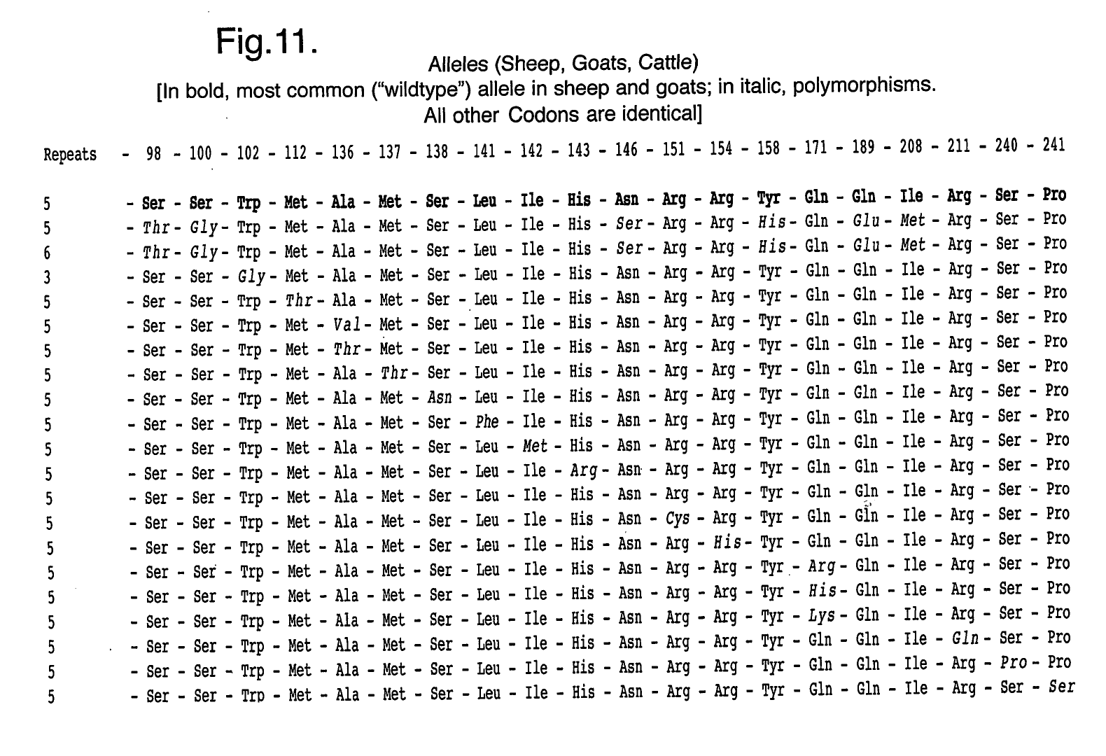

The sequence of ovine and bovine prion protein is shown hereinafter in FIGS. 10 and 11.

The expression “epitopic region” refers to any fragment of the basically defined sequence, which gives rise to an antigenic response.

In an alternative embodiment, the method involves comparison of the amounts of a peptide sequence within a particular cell type of a test animal, wherein the concentration is characteristic of a particular strain of TSE, with results obtained in a similar manner from cells of at least one comparative animal suffering from a known strain of TSE. Preferably the comparative animal will be of a similar breed to the test animal, in order to prevent any difference resulting from genotype of the animal in the processing. As illustrated hereinafter however, in some cases the genotype has little influence on the results obtained.

It has been found for example, that BSE-agent-infected sheep could be differentiated from natural-sheep scrapie by the higher levels of intra-neuronal PrP accumulation in brain detected by labelling for a range of PrP peptide sequences.

For example, in sheep, it has been found that a peptide sequence which binds to an antibody raised to a peptide corresponding to amino acids 84-105 of the prion protein or an epitopic region thereof (for example, a peptide corresponding to amino acids 89-104 of sheep is concentrated in neuronal cells differently in scrapie and/or a bovine spongiform encephalopathy (BSE) derived strain, and that this difference can be used to type a strain.

Similarly, a peptide sequence which binds to an antibody raised to a peptide corresponding to amino acids 217-231 of the prion protein of cattle or an epitopic region thereof, is distributed differently in neuronal cells of sheep infected with natural scrapie as compared to a bovine spongiform encephalopathy (BSE) derived strain.

In yet a further alternative, it has been found that a peptide sequence which binds to an antibody corresponding to amino acids 54-60 of sheep prion protein or an epitopic region thereof, and is concentrated differently in the extracellular neuropil in sheep with scrapie as compared to bovine spongiform encephalopathy (BSE) derived strain.

The results reported herein suggest that there is strain dependent processing of disease specific PrP in particular cell types within the nervous system and LRS which can be used to differentiate between BSE agent and other scrapie strain infections of sheep.

Suitably, the presence or concentration of a peptide sequence within particular cell type may be determined using conventional immunohistochemical techniques. In particular, the method of the invention will use an antibody or specific binding fragment thereof, which is specific for said peptide sequence. These may be applied for example in a conventional ELISA format.

In the work reported hereinafter, the sequence to which the antibodies were raised is known for most of the antibodies used in this study. However, the precise epitopes recognised by each antibody may not always be identical to the sequence of the immunising peptide. A detailed analysis of one of the most important antibodies used in this study (521.7) (Garssen et al. 2000, Microscopy Research and Technique. 50, 32-39) suggests that this antibody has a wider sequence of immunoreactivity than would be predicted from the source immunogen. This antibody appears to react with GG, GS and GQ di-peptides over the amino-acid range 33-146 of the PrP protein. In particular epitopes between 84 and 105 are strongly recognised. Similarly, although the source immunogen of FH11 and BG 4 are 54-60 a personal communication cited in (Foster et al. 2001, Journal of General Virology 82, 267-273) suggest that the epitopes recognised by BG4 and FH11 are 47-57 and 89-99. These results would suggest that the affinity of these antibodies for the ovine sequence 89-99 are not retained in formalin fixed tissues.

The method of the invention may be carried out as a cellular assay as illustrated hereinafter. However, it may be preferable to modify the techniques so that they can be effected in a biochemical environment. This may require that the various cell types cells are separated prior to detection of the peptide sequence. Conventional separation methods such as flow cytometry may be employed.

Once separated, cells may be lysed in a known manner and the contents probed for the presence or amount of the target peptide sequence, for example using an ELISA. If convenient, antibodies may be immobilised on support media such as beads or in wells, prior to detection of binding.

The invention further provides a kit for typing a strain of a transmissible spongiform encephalopathy (TSE) using a method as described above, said kit comprising an antibody or a binding fragment thereof which is specific for a peptide sequence derived from a TSE which is found within a cell of a particular cell type of an infected animal and wherein the presence or concentration of said peptide sequence within said cell type is characteristic of a particular strain of TSE, and means for detecting said antibody.

The means for detecting the antibody may be secondary antibodies, which may be labelled, for example with a fluorescent label.

The invention will now be particularly described by way of example with reference to the accompanying figures in which:

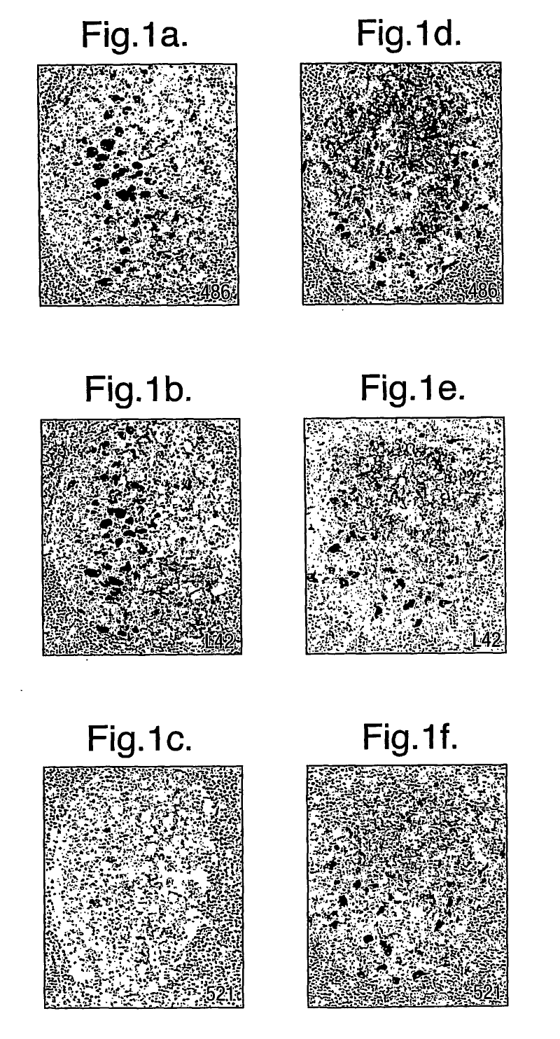

FIG. 1 shows a comparison of retropharyngeal lymph node from sheep infected with scrapie or BSE labelled with three antibodies. Serial sections of the same secondary follicle taken from a group1 BSE agent infected Romney sheep (a-c) and similarly, serial sections from the same secondary follicle of a scrapie infected Suffolk sheep (d-f) labelled with antibodies R486 (d), L42 (e) and 521.7 (f). In the latter sheep there is labelling of the light zones with marked-FDC type labelling and also an intense granular labelling associated with individual cells (TBM) located in the light zone, dark zone and follicular mantle. In the follicle from the BSE agent infected sheep there is a similar pattern with the R486 (a) and L42 (b) but with the 521.7 antibody there is almost exclusively a mild FDC type pattern with little or no evidence of TBM labelling (c).

a-f x 120;

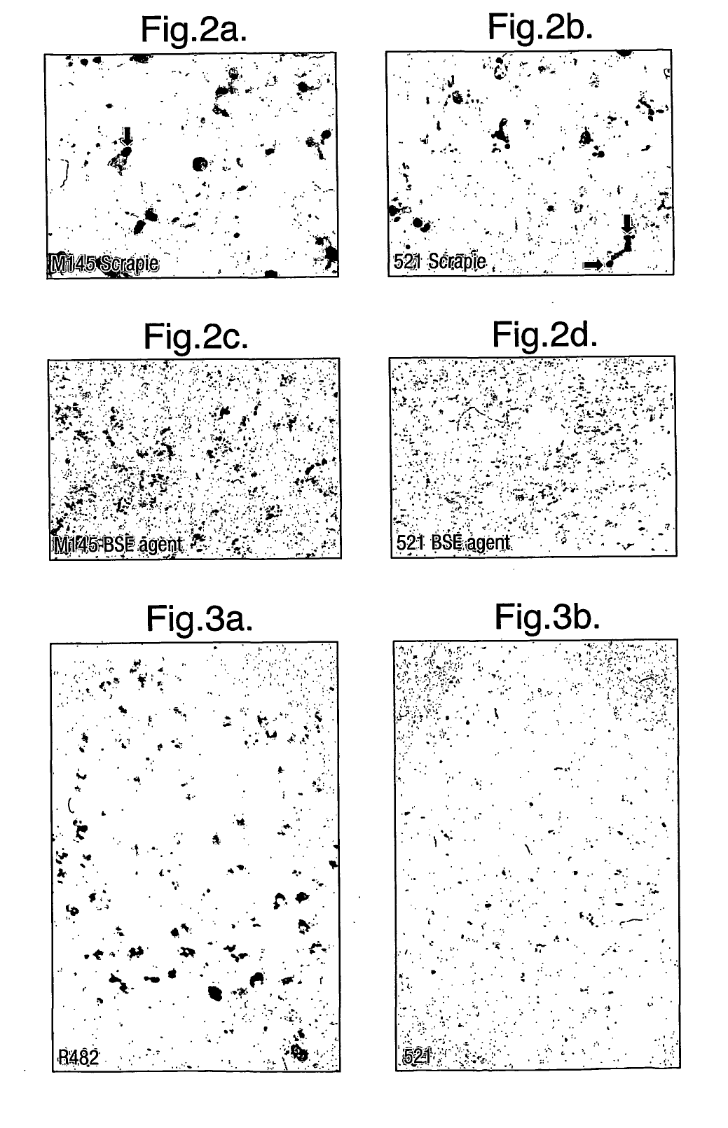

FIG. 2 shows serial sections of a secondary lymphoid follicle from the retropharyngeal lymph node of a sheep in group 2 sheep killed at 10 months post challenge. With antibody R486 there is a widespread focally intense granular pattern of immunolabelling consistent with a TBM location. However there is no significant immunolabelling of FDC (a). In the serial section treated with 521.7 there is only sparse labelling of indeterminate cellular pattern (b).

a-b x 250;

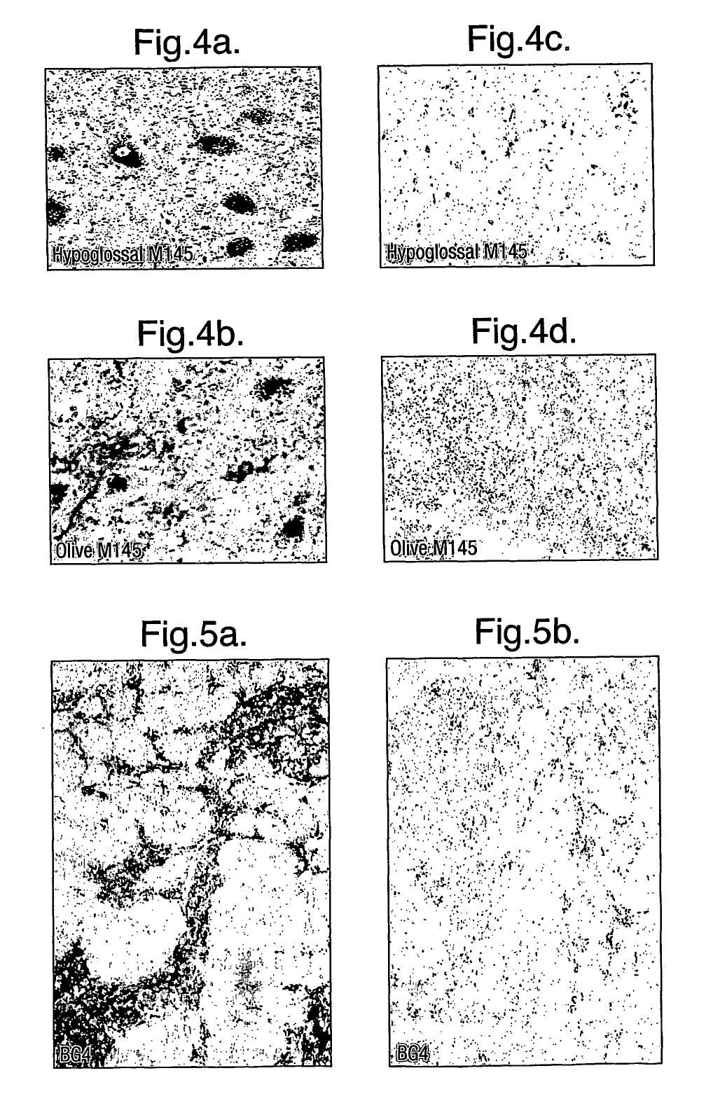

FIG. 3 shows a comparison of brain from sheep infected with BSE or scrapie agent and treated with antibody R145. Sections are through the hypoglossal and olivary nuclei at the obex of clinically affected sheep from groups1 and 4. Note there is intense granular intracytoplasmic labelling of neurones in the hypoglossal (a) and olivary (b) nuclei in a sheep infected with BSE agent. By comparison note that there is only weak intracytoplasmic intra-neuronal labelling present in the same nuclei from a sheep infected with scrapie (c,d).

a x220; b x 280; c x 230; d x 190 c and x 250;

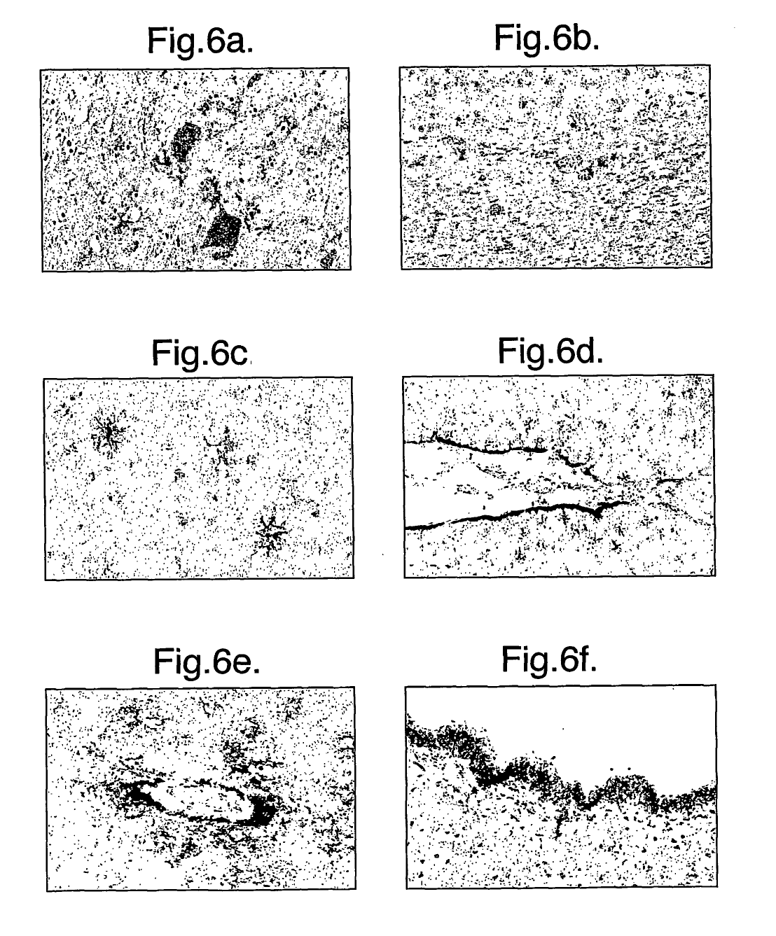

FIG. 4 shows a comparison of sections of brain from a sheep infected with BSE agent or scrapie agent and treated with antibody BG4. Note that there is coarse particulate and stellate patterns of intense labelling in a section through the spinal tract nucleus of the trigeminal nerve (a) from a sheep infected with BSE (group1). In contrast, the amount of labelling in the same area of a scrapie brain (group 6) is considerably less when labelled with this antibody (b).

a-b x 100; and

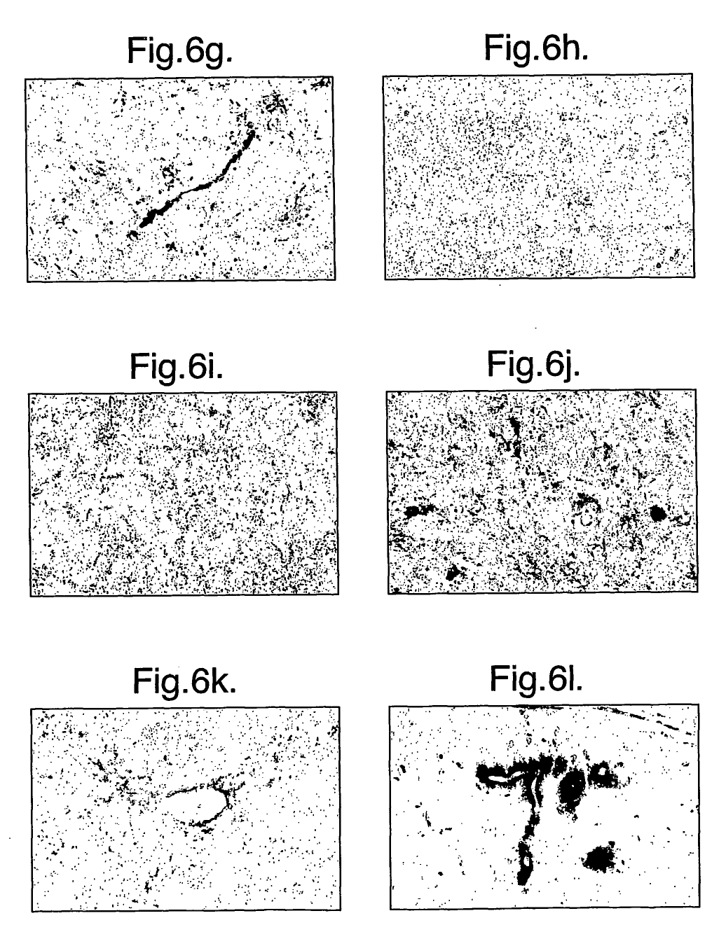

FIG. 5 shows a comparison of the labelling of glial cells in sections of brain from sheep infected with BSE or scrapie and treated with antibodies R145 and 521.7.

Dense granular labelling can be seen adjacent to cellular nuclei, morphologically consistent with those of astrocytes and microglia, (arrows a, b) in a (group 8) sheep with scrapie. a and b are adjacent sections, through the olivary nucleus, treated with R145 and 521.7 antibodies respectively. Similar labelling was seen in sheep infected with R145 antibody. Antibody 521.7 (as with FH11 and BG4) did not label intra-glial granules (d). a,b x750; c,dx 350

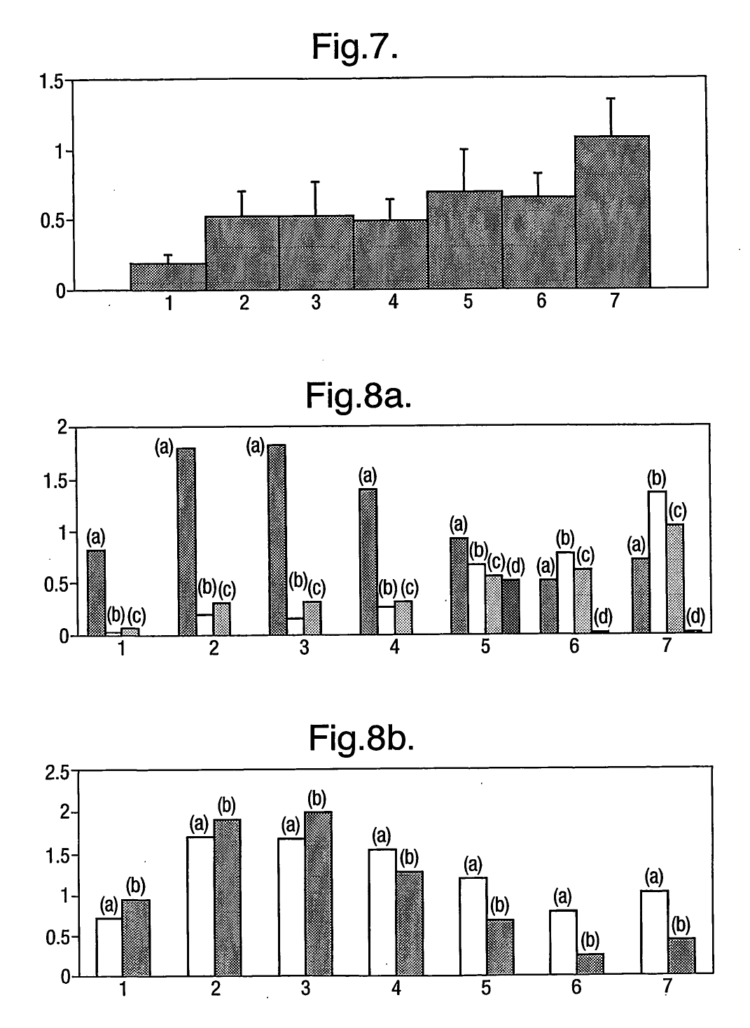

FIG. 6a: Intraneuronal type: accumulation of granular deposits of PrP^{d} in the perikarya of neurons of the red nucleus. VRQ/ARQ Shetland sheep. ABC Immunoperoxidase with R-486 antibody and haematoxylin counterstain x2160.

FIG. 6b: Intraglial type: accumulation of coarse granular deposits of PrP^{d} in the cytoplasm of glial cells in the cerebellar white matter. SSBP/1-challenged VRQ/ARQ Cheviot sheep. ABC Immunoperoxidase with R-486 antibody and haematoxylin counterstain x2160.

FIG. 6c: Stellate type: branching deposits of PrP^{d} on the processes of astrocytes in the cerebellar cortex. ARQ/ARQ Suffolk sheep. ABC Immunoperoxidase with R-486 antibody and haematoxylin counterstain x2160.

FIG. 6d: Subpial type: continuous loose mesh of PrP^{d} underneath the pia matter in the cerebral cortex. VRQ/ARQ Shetland sheep. Note concurrent stellate type. ABC Immunoperoxidase with R-486 antibody and haematoxylin counterstain x1080.

FIG. 6e: Perivascular type: thick, strongly labelled PrP^{d} accumulation around a blood vessel in the cerebral white matter. ARQ/ARQ Suffolk sheep. ABC Immunoperoxidase with R-486 antibody and haematoxylin counterstain x2160.

FIG. 6f: Subependymal type: continuous, strongly labelled mesh of PrP^{d} underneath the ependyma of the lateral ventricles at the level of the striatum. ARQ/ARQ Suffolk sheep. ABC Immunoperoxidase with R-486 antibody and haematoxylin counterstain x2160.

FIG. 6g: Linear type: thick thread-like deposits of PrP^{d} in the neuropil at the level of the obex. VRQ/ARQ Shetland sheep. ABC Immunoperoxidase with R-486 antibody and haematoxylin counterstain x2160.

FIG. 6h: Fine punctate type: powdery, diffuse PrP^{d} accumulation in the neuropil at the level of the rostral medulla oblongata. VRQ/ARR Cheviot sheep. ABC Immunoperoxidase with R-486 antibody and haematoxylin counterstain x2160.

FIG. 6i: Coarse particulate type: irregular, conspicuous deposits of PrP^{d} in the neuropil at the level of the midbrain. ARQ/ARQ Suffolk sheep. ABC Immunoperoxidase with R-486 antibody and haematoxylin counterstain x2160.

FIG. 6j: Coalescing type: amorphous, strongly labelled masses of Prp^{d} in the neuropil at the level of the obex. Note concurrence with coarse particulate deposits. VRQ/ARQ Shetland sheep. ABC Immunoperoxidase with R-486 antibody and haematoxylin counterstain x2160.

FIG. 6k: Perineuronal type: thin deposits of PrP^{d} around the plasmalema of a neurone in the fastigial nucleus of the cerebellum. ARQ/ARQ Suffolk sheep. ABC Immunoperoxidase with R-486 antibody and haematoxylin counterstain x2160.

FIG. 6l: Vascular plaques: radiate, fibrillar accumulations of PrP^{d} around blood vessels in the cerebellar cortex. Note also intramural deposits. VRQ/VRQ Welsh Mountain sheep. ABC Immunoperoxidase with R-486 antibody and haematoxylin counterstain x2160;

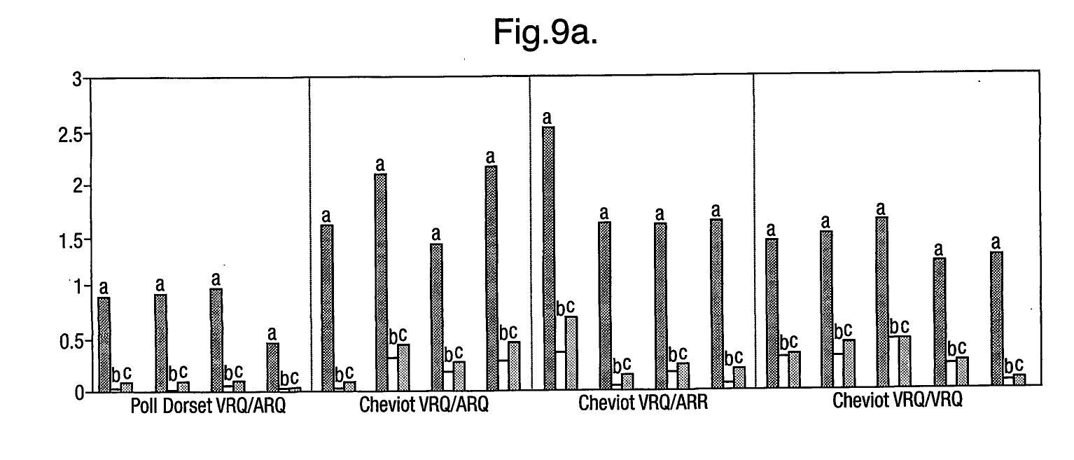

FIG. 7: Magnitude of global PrP^{ds} accumulation in the different sheep groups under study. 1: SSBP1-infected Poll Dorset VRQ/ARQ; 2: SSBP1-infected Cheviot VRQ/ARQ: 3: SSBP1-infected Cheviot VRQ/ARR; 4: SSBP1-infected Cheviot VRQ/VRQ; 5: Naturally-infected Welsh Mountain VRQ/VRQ; 6: Naturally-infected Shetland-Cheviot VRQ/ARQ; 7: Naturally-infected Suffolk ARQ/ARQ. Bars express mean values and lines standard deviation;

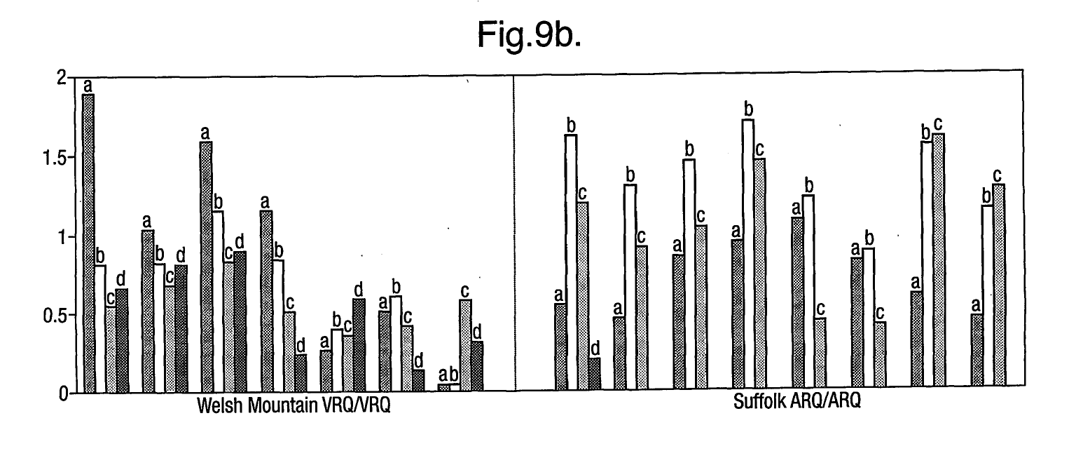

FIG. 8a: PrP^{d} profiles of the different sheep groups studied (for identification of the groups refer to Table 6). (a): intracellular PrP^{d}. (b): astrocyte-associated PrP^{d}. (c): neuropil PrP^{d}. (d): vascular PrP^{d}.

FIG. 8b: Intracelluar PrP^{d} profiles of the different sheep groups studied (for identification of the groups refer to Table 6). (a): intraneuronal PrP^{d}. (b): intraglail PrP^{d};

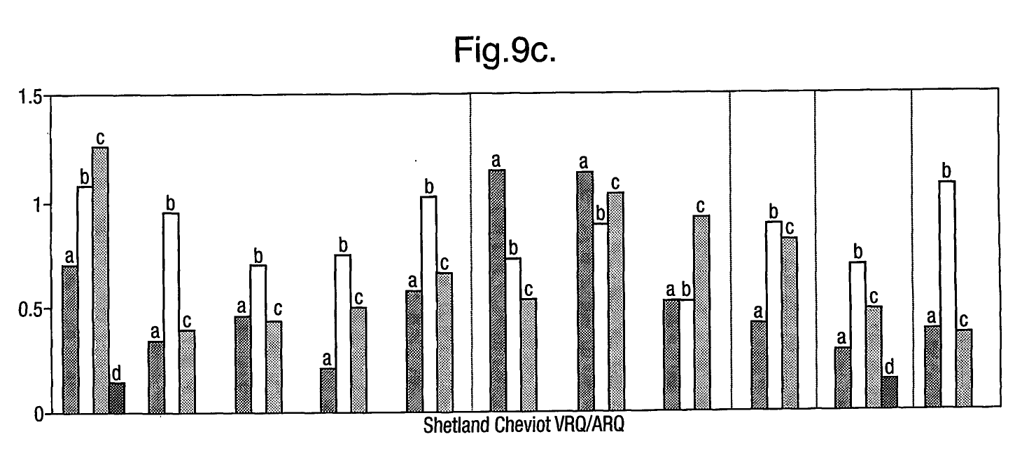

FIG. 9a: PrP^{d} profiles of individual sheep challenged with the SSBP1 strain of scrapie (group 1 to 4). (a): intracellular PrP^{d}. (b): astrocyte-associated PrP^{d}. (c): neuropil PrP^{d}.

FIG. 9b: PrP^{d} profiles of naturally-affected individual sheep (groups 5 and 7, one flock each). (a): intracellular PrP^{d}. (b): astrocyte-associated PrP^{d}. (c): neuropil PrP^{d}. (d): vascular PrP^{d}.

FIG. 9c: PrP^{d} profiles of naturally-affected individual sheep (group 6, five different flocks separated by dotted lines). (a): intracellular PrP^{d}. (b) astrocyte-associated PrP^{d}. (c): neuropil PrP^{d}. (d): vascular PrP^{d}.

FIG. 10 shows a comparison between ovine and bovine PrP protein sequences (most common alleles);

FIG. 11 summarises allelic variants found in the ovine sequence, where the most common “wildtype” is shown in bold type.

### EXAMPLE 1

### Immunohistochemical Differences between Natural Scrapie or the Bovine Spongiform Encephalopathy Agent in Sheep

A range of antibodies (Table 1) generated to various peptide sequences of the PrP protein were obtained and were used to compare the tissue and cellular patterns of disease-specific PrP labelling in brain and in the LRS from sheep infected either with natural scrapie or with bovine spongiform encephalopathy (BSE) agent.

Eight groups of sheep were available for study (Table 2). In groups 1-3 Romney-Marsh or Suffolk sheep were orally challenged with 5 g of a pool of BSE brains and groups of five PrP^{ARQ/ARQ} sheep sequentially killed. Brains and viscera were obtained from groups of five sheep at 4 or 6 monthly intervals. The earliest onset of clinical disease in BSE challenged Romney sheep was at 20 months post inoculation. Further details of the Romney sheep, tissues collected and distributions of PrP have been described (Jeffrey et al., 2001a, Oral BSE challenge of sheep:1: Onset and distribution of disease specific PrP accumulation in brains and viscera of Romney sheep. Journal of Comparative Pathology (In press)).

Groups 4-8 consisted of natural scrapie cases. Group 4 sheep consisted of four clinical cases of Suffolk scrapie obtained from a farm in Scotland. Group 5 sheep were obtained from this same heavily infected source farm and consisted of a further ten PrP^{ARQ/ARQ} genotype Suffolk sheep which were sequentially tonsil biopsied at 4, 10, 14, 20 and 26 months. In addition serial necropsies were performed on 12 pre-clinical cases. Tissues from all of these animals were available for examination. Details of this flock and the results for pre-clinical testing for disease specific PrP have been described (Jeffrey et al., 2001b, A preliminary investigation of scrapie peripheral pathogenesis in Suffolk sheep using sequential necropsies and tonsil biopsies. Journal of Comparative Pathology In Press). Group 6 consisted of clinical scrapie cases from three farms in a confined geographical area (17 Shetland cross sheep of genotypes PrP^{ARQ/ARQ}; PrP^{VRQ/VRQ}; PrP^{ARR/VRQ}) while Group 7 cases came from a single source in Wales (3 Welsh mountain sheep with a genotype of PrP^{VRQ/VRQ}). Finally, a miscellany of 12 clinical cases of 2 genotypes originating from several widely dispersed UK source locations made up group 8 sheep.

### Tissues and Procedure

Brains and a range of lymphoid tissues (spleen, tonsil, pre-scapular lymph node, mesenteric lymph node, retropharyngeal lymph node, mediastinal lymph node, spleen, and gut associated lymphoid tissue obtained mainly from the ileum and colon) were obtained at elective necropsy and fixed in 10% neutral phosphate buffered formalin, trimmed, post-fixed and embedded according to standard procedures. For the purposes of this study examination of brain tissue was confined to the level of the obex.

Tissue sections, 5μ thick, were cut on a microtome and mounted on treated glass slides (superfrost plus, Menzel-Glaser, Germany) and dried overnight at 37° C. Sections were de-waxed and hydrated according to conventional protocols and then subjected to an antigen retrieval procedure. Sections were immersed in 98% formic acid for 5 min, washed in running tap water and then immersed in 0.2% citrate buffer and autoclaved for 5 min at 121° C. Two initial blocking step, to quench endogenous peroxidase activity (3% hydrogen peroxide for 20 min) and to remove non-specific tissue antigens (5% normal horse serum for 60 min) were performed at room temperature. Incubation with the primary antibody was performed overnight at 4° C. following a rinse in tris buffer bound antibody was labelled by means of a commercial immunoperoxidase technique (Vector-elite ABC; Vector Laboratories, Peterborough). After a further rinse sections were immersed in 0.5% copper sulphate to enhance the intensity of the immunoperoxidase reaction product and counterstained with Mayer's haematoxylin for 2 minutes.

A range of primary antibodies was selected to label amino acid sequences spanning the PrP protein from the N terminal domain of the flexible tail (Riek et al., 1997, NMR characterization of the full-length recombinant murine prion protein, mPrP(23-231). FEBS Letters 413, 282-288), through the globular domain of the protein (Riek et al., 1997, NMR characterization of the full-length recombinant murine prion protein, mPrP(23-231). FEBS Letters 413, 282-288) to the carboxyl terminus. The antibodies used and the sequence to which they were raised (where known) are shown in Table 1.

All the antibodies directed to the globular domains and the C termini of the PrP molecule (approximately from amino-acid 120) gave essentially the same results and are therefore referred to as a group. As none of the antibodies used was able to discriminate between the normal and abnormal isoforms of PrP, but the animals in the study were all known to be infected with TSE, it has been assumed that positive label in tissue sections is a result of disease specific PrP accumulation.

The intensity of labelling was judged subjectively according to the following criteria; − no staining; +/− trace staining only visible at high magnification (x40 objective) as occasional light brown labelling; + staining visible at moderate (x10 objective) magnifications as light brown label; ++ brown label visible in most of target areas(s) visible at x2.5 objective; +++ dark brown label over most of the target site(s); ++++ intense uniform brown/black label visible at the lowest magnifications (x2.5) over all the target area (s).

### Results

LRS tissues from Clinically Diseased Sheep In LRS tissues most antibodies (Table 3 FIG. 1) labelled both FDC and TBM in Group 4/5 Suffolk sheep scrapie and of BSE agent infected Groups 1-3 sheep. In general, the labelling of TBM in BSE agent groups 1-3 lymph node infected sheep was slightly more intense than that in scrapie. Only two antibodies (FH11, BG4) did not label PrP either scrapie or BSE agent affected TBM.

Another two antibodies (521.7, P4) labelled TBM from BSE-agent-affected animals less than FDC in the same section. With the 521.7 antibody a virtual absence of the TBM associated pattern of labelling was invariably found for BSE agent infected lymphoid tissues but not for scrapie infected secondary follicles sheep where, with the same antibody, TBM labelling was more intense than that of FDC labelling in the same section.

In order to determine whether the almost complete absence of 521.7 labelling of TBM was confined to BSE agent infections alone, a selection of scrapie affected sheep of different genotypes, breeds and geographical origins within the UK were examined. The BSE agent and scrapie sources tested are shown in Table 2. To determine whether the different cellular patterns of immuno-labelling were site specific a range of LRS tissues including spleen, tonsil and lymph nodes from the sources shown in Table 2 were examined. The same relative intensity of TBM and FDC labelling were found for all the LRS sites examined. At each LRS tissue site of group1 BSE agent infected sheep TBM labelling with the 521.7 antibody was diminished or lost when compared with other antibodies whereas good TBM labelling was found for all antibodies tested including 521.7 in all LRS tissues in all of the genotypes of scrapie affected sheep.

Lymphoid Tissues from Pre-clinical Disease

To determine whether the differential labelling of TBM described above was also present before the onset of clinical disease, sheep from groups 2,3 and 5 were examined at time points shown in Table 2. As previously described, in the early stages of both BSE agent infection (Jeffrey et al. 2001a, Oral BSE challenge of sheep:1: Onset and distribution of disease specific PrP accumulation in brains and viscera of Romney sheep. Journal of Comparative Pathology (In press)) and natural scrapie (Jeffrey et al. 2001b, A preliminary investigation of scrapie peripheral pathogenesis in Suffolk sheep using sequential necropsies and tonsil biopsies. Journal of Comparative Pathology In Press), disease specific PrP was initially detected as sparse accumulations within TBM. In BSE infected sheep from groups 2 and 3 at up to 16 months post inoculation neither TBM nor FDC could be identified using 521.7 antibody (FIG. 2) but TBM containing PrP accumulations were detected using other (R486/R482/R145) antibodies (FIG. 2). In contrast, 521.7 labelled TBM in pre-clinical scrapie cases. These differences between disease specific B$E-agent and scrapie PrP labelling patterns were found at all the pre-clinical stages of BSE-agent-infected sheep examined at 4, 10, 16 and 22 months post dosage when compared with the pre-clinical scrapie infected Suffolk sheep at 12, 14,16 and 20 months of age.

### CNS

The patterns of PrP immunolabelling obtained following application of several antibodies on the medulla oblongata of BSE agent infected sheep were compared with those of scrapie infected sheep of various breeds and genotypes as shown in Table 4.

Two antibodies, FH11 and BG4, did not label intracellular disease specific PrP in the brains of sheep in groups 1-8. However, both of these antibodies produced strong coarse, particulate and stellate patterns of extracellular neuropil immunolabelling. In contrast, antibody 521.7 showed affinity for intracellular neuronal accumulations of PrP with only moderate or weak labelling of extracellular PrP accumulations. Antibodies to amino-acids near the C terminus such as antibody R145 showed an affinity for both intra-cellular and extracellular deposits but did not give as high an intensity of labelling in either of these broad patterns of labelling as did BG4 or FH11. The P4 antibody detected intra-neuronal labelling and variable levels of stellate, fine punctate and sub-ependymal labelling.

When the cellular patterns and neuroanatomical distributions of PrP in BSE and scrapie are compared, marked differences were found in both the intensity and distribution of intra-neuronal PrP. In all BSE-agent infected brains from group 1 and brains of 16-22 month post-dosed sheep of group 2, a strong and widespread granular intra-neuronal PrP labelling was observed when antibodies recognising the globular domains or the C terminus of the PrP protein were used.

In scrapie brains, disease specific intra-neuronal PrP was detected at lower levels than that of BSE affected brains including with antibodies R145, R486 and 521.7 (FIG. 3, Table 4). Only one antibody when applied to scrapie brains (P4) gave greater intra-neuronal labelling, particularly in the olives, (granular deposits) when compared with BSE agent infection (diffuse intra-neuronal labelling). In a minority of scrapie infected sheep of PrP^{VRQ/VRQ} and PrP^{VRQ/ARQ} genotypes from groups 6 and 8, more widespread intra-neuronal PrP accumulations were found but in all of these brains the overall amount of disease specific PrP labelling was weak. In addition to the differences in intra-neuronal labelling, an increase in the proportion of coarse particulate neuropil labelling was found in BSE agent infected brains in groups 1 and 2 when compared with sheep scrapie from groups 4-8 especially with the BG4 and FH11 antibodies (FIG. 4). In contrast, sheep scrapie brains in the latter groups showed an increased proportion of the stellate pattern compared with BSE agent infected brains with the same antibodies.

Findings similar to the LRS system were found in respect of intra-glial PrP accumulation in brain tissue (FIG. 5). Intra-glial immunolabelling was seen in all BSE cases and in a proportion of scrapie brains. BSE agent infected brains did not show intra-glial PrP accumulation when treated with 521.7 antibody. However, antibodies such as R486, R482 and R145 allowed the demonstration of marked intra-glial PrP accumulation. Intra-glial PrP accumulation was rarely observed in group 5 and was an inconsistent feature of scrapie infected brains from groups 6-7. Where present, scrapie associated intra-glial PrP showed good immunolabelling with all antibodies tested other than FH11 and BG4.

To determine whether the subjectively assessed differences in staining of CNS tissue described above could be used to discriminate between BSE agent infected brains and natural scrapie a miscellany of twelve scrapie cases were selected (Groups 7-8) and mixed with three clinical (Group 1) and two pre-clinical (Group 2) cases of BSE agent infected sheep brains. Sections of medulla were then cut, blind-coded and labelled with 8 different antibodies. Using the comparative intensity and distribution of intra-neuronal labelling following staining with P4 and R145/R486/521.7 four of the BSE cases were identified and the final pre-clinical BSE case was classified as of unknown type when results were de-coded.

In summary therefore, taking the findings on the LRS, BSE agent infection in sheep groups 1-3 could be differentiated from all natural sheep scrapie sources (Groups 4-8) based on the pattern and distribution of FDC and TBM labelling when tissues were labelled with two of the following peptide specific antibodies: R145, 521.7. In CNS tissues BSE agent infected sheep obexes could be recognised by

1. the intensity and distribution of intra-neuronal labelling using P4 and R145/R 486/521.7

2. the intensity of immunolabelling with BG4/FH11 and the relative proportions of stellate labelling versus coarse particulate granular neuropil labelling in the spinal tract of the trigeminal nerve.

3. and the absence of glial labelling with 521.7 antibody compared with antibodies to the globular domains/C-terminus of PrP.

The intra-cellular accumulation of glial PrP in mice is found at sub-cellular levels in astrocytic and microglial lysosomes (Jeffrey et al., 1994, Correlative light and electron microscopy studies of PrP localisation in 87V scrapie. Brain Research 656, 329-343) and in lymphoid tissues within TBM lysosomes (Jeffrey et al., 2000, Sites of prion protein accumulation in scrapie infected mouse spleen revealed by immuno-gold electron microscopy. Journal of Pathology. 191, 323-332). It has previously been shown that extracellular PrP deposited in the brains of scrapie affected mice and sheep is present as the full length protein (Jeffrey et al., 1996,. Neurodegeneration 5, 101-109; Jeffrey et al,. 1998, Veterinary Record, 142, 534-537). However, intra-lysosomal PrP will be acted upon by several protein degrading enzymes it is possible that intra-lysosomal accumulations of PrP may be truncated. Both BG4 and FH11 antibodies recognise domains in the N-terminal segments of the flexible tail of the PrP protein (Reik et al., 1997) which are removed on protease K treatment of the scrapie isoform of PrP.

Thus, the absence of PrP in FH11 or BG4 labelled TBMs and glial cells of both scrapie and BSE agent origins might be the result of in-vivo N-terminal truncation within lysosomes.

In contrast the extracellular forms of disease specific PrP including that released around neurones, astrocytes and FDC are present as the full length protein (Jeffrey et al., 1996, Neurodegeneration 5, 101-109) and therefore label with antibodies which recognise both the protease resistant and protease sensitive domains of disease specific PrP. Furthermore the differences in the labelling affinity of specific antibodies to accumulated PrP in some BSE agent infected cells compared with that of sheep scrapie suggests that there may be different processing or truncation of BSE agent specific PrP.

Without being constrained by mechanistic considerations, it is hypothesised that conformational changes result in a variable but longer N terminal deletion of the disease specific form of PrP that accumulates following BSE agent infections of sheep. This makes it possible to distinguish between scrapie and BSE agent infection of peripheral lymphoreticular tissues and brain. As the 521.7 antibody recognises epitope(s) between 84-105 it is possible that truncation of PrP between these residues results in abrogation of immunolabelling in BSE agent affected tissue but retention of labelling in scrapie where the truncation might be expected to be near to codon 82 as suggested for type1 human PrP^{res} (Parchi et al., 2000, Genetic influence on the strucutral variations of the abnormal prion protein. Proceedings of the National Academy of Sciences, USA 97, 10168-10172).

The method of the invention may be applicable in a wide variety of TSEs. Detailed investigations across a range of sites in brain have indicated, that ‘PrP profiling’ might be effective in determining other naturally occurring strains or isolates present in individual sheep brains. As in visceral labelling of PrP, there is also some evidence that the type of PrP^{res} found in B$E agent infected animal brains might also be different from that formed in brain of natural sheep scrapie. Although moderate immunohistochemical labelling of PrP in glial cells of scrapie brains occurs in only a proportion of scrapie cases, where this did occur there was little difficulty in recognising scrapie infection because of the assumed shorter N terminal fragment size of digested scrapie associated PrP accumulations in glia when compared with BSE agent associated PrP in glia. Secondly, the overall magnitude and proportions of stellate and particulate labelling were different. Thirdly, in sheep scrapie intra-neuronal labelling with P4 antibody was always greater (especially in the olives) than with other antibodies such as R145/R482/R486/L42 but the converse was observed in BSE agent infected brains (Table 4). These relative differences between intra-glial and intra-neuronal labelling of BSE affected brains and natural scrapie affected brains is consistent with the idea that the disease specific form of BSE PrP is synthesised and/or folded differently from disease specific scrapie PrP.

Furthermore, at least one antibody (P4) detected differences between PrP present in scrapie and BSE brains but not in the LRS suggesting that that the precise differences in the strain specific processing of PrP is different for different cells or groups of cells. This notion has some biochemical support as there are differences in both PrP^{res} and PrP^{sen} extracted from viscera or brain of the same animal (Rubenstein et al.,1991, Journal of Infectious diseases 1.64, 29-35; Somerville et al., 1997b, Journal of General Virology 78, 2389-2396; Somerville, 1999, Journal of General Virology 80, 1865-1872).

TABLE 1
Details of antibodies used and their specificity for epitopes.

| | | | Species/ |
|---|---|---|---|
| | | | sequence of |
| Antibody | | | immunogen/ |
| code | Type | Reference (where relevant) | source |
| IB3 | Rabbit | (Farquhar et al., 1989 | 18-19; 35-44; |
| | polyclonal | Virology Methods | 129-132. |
| | | 215-222; Garssen et al., | Murine |
| | | 2000, Microscopy | |
| | | Research and | |
| | | Technique 50, 32-39) | |
| R24 | Rabbit | (Caughey et al., 2001, | 22-37 hamster |
| | polyclonal | Journal of Virology, 65, | |
| | | 6597-6603) | |
| BG4 | Mouse | Foster et al., (2001) J. | 54-60 bovine |
| | monoclonal | Gen. Virol. 82, 267-273 | recombinant |
| FH11 | Mouse | Foster et al., (2001) | 54-60 ovine |
| | monoclonal | supra. | |
| P4 | Mouse | (Hardt et al., 2000, J. | 89-104 ovine |
| | monoclonal | Comp. Pathology, 122, | |
| | | 43-45) | |
| 521.7 | Rabbit | (Van Keulen et al., 1996, | 94-105 ovine* |
| | polyclonal | J. Clin. Micriobiol. 34, | |
| | | 1228-1231) | |
| 505 | Rabbit | (van Keulen et al., 1996) | 100-111 ovine |
| | polyclonal | supra. | |
| 1A8 | Rabbit | Langeveld et al., 1993, | 102-126 bovine |
| | polyclonal | Antigenic sites of bovine | 126-143 |
| | | prion protein, 315-321. | 152-194 |
| | | Brussels) | 199-244 |
| L42 | Mouse | Hardt et al., 2000 | 145-163 |
| | monoclonal | | |
| 532 | Rabbit | (van Keulen et al., | 145-177 ovine |
| | polyclonal | 1996) supra. | |
| R486 | Rabbit | | 217-231 bovine |
| | polyclonal | | |
| R482 | Rabbit | | 217-231 bovine |
| | polyclonal | | |
| R145 | Rat | | 217-231 bovine |
| | monoclonal | | |
| R476 | Rabbit | | 219-234 Bovine |
| | polyclonal | | |
| 523.7/ | Polyclonal | (van Keulen et al., 1996) | 219-234 ovine |
| 524 | | supra. | |
| IB4 | Rabbit | | Undetermined |
| | polyclonal | | |

TABLE 2
Breed, genotype and clinical status of
sheep available for examination.

| | | | | No. | | | 521.7 |
|---|---|---|---|---|---|---|---|
| | Agent | | | in | Clinical | Pre- | TBM |
| Group | or source | Breed | Genotype | test | disease | clinical | pattern^{#} |
| 1 | BSE | Romney* | ARQ/ARQ | 4 | 22-26 m | | − |
| 2 | BSE | Romney* | ARQ/ARQ | 12 | — | 10-22 m | − |
| 3 | BSE | Suffolk* | ARQ/ARQ | 8 | — | 4-10 m | − |
| 4 | Natural | Suffolk | ARQ/ARQ | 4 | 23-26 m | | + |
| 5 | Natural | Suffolk** | ARQ/ARQ | 12 | — | 8-20 m | + |
| 6 | Natural | Shetland | VRQ/ARQ | 12 | 30-48 m | — | + |
| | | ShetlandX | VRQ/VRQ | 1 | | | |
| 7 | Natural | Welsh | VRQ/VRQ | 3 | 26-36 m | — | + |
| | | mountain | | | | | |
| 8 | Natural | Various | ARQ/ARQ | 10 | 30-60 | — | + |
| | | Various | VRQ/ARQ | 2 | | | |

^{#}+ or − indicates whether TBM-like patterns of granular immunolabelling of various lymphoid tissues were observed when treated with antibody 521.7

TABLE 3
Immunolabelling pattern of scrapie and BSE agent
infected sheep lymphoreticular tissues reacted
with selected antibodies.

| | Tissue | Antibody | | | | | | | | | |
|---|---|---|---|---|---|---|---|---|---|---|---|
| Strain | pattern | FH11 | BG4 | 521.7 | P4 | 505 | L42 | R482 | R486 | R145 | 523.7 |
| BSE | FDC | + | + | +++ | +++ | + | +++ | +++ | ++ | + | +++ |
| | TBM | − | − | +/− | ++ | +++ | ++++ | ++++ | ++++ | ++++ | ++++ |
| Scrapie | FDC | + | + | +++ | +++ | +/− | +++ | +++ | +++ | + | +++ |
| | TBM | − | − | +++ | ++ | ++ | +++ | +++ | +++ | +++ | ++++ |
| Amino-acids | | 55-65 | 55-65 | 84-105 | 89-104 | 100-111 | 145-167 | 217-234 | 217-234 | 217-234 | 219-232 |
| recognised | | | | | | | | | | | |

− +, ++, +++, ++++. Increasing intensity of immunolabelling; for definitions see materials and methods.

Only some of the antibodies tested are shown. Shaded area indicates those antibodies that produce results of a different pattern from the majority. All these antibodies recognise the flexible tail of PrP. Section intensely shaded is the key antibody for differentiating between BSE agent infection of sheep and scrapie.

TABLE 4
showing the intensity of intra-neuronal labelling in
brain medulla of a Suffolk scrapie and BSE
agent infected Romney sheep.

| | | Antibody and epitope recognised | | | | | | | | | |
|---|---|---|---|---|---|---|---|---|---|---|---|
| Strain | | FH11 | BG4 | 521.7 | P4 | 505 | L42 | 482 | 486 | R145 | 523.7 |
| breed | Nucleus | 54-60 | 54-60 | 84-105 | 89-104 | 100-111 | 145-167 | 217-231 | 217-231 | 217-231 | 219-232 |
| BSE/ | Cuneate | − | − | ++ | ++ | +++ | +++ | +++ | +++ | +++ | +++ |
| Romney | XII | − | − | +++ | + | +++ | + | +++ | +++ | ++++ | +++ |
| | DMNV | − | − | + | + | + | +/− | ++ | ++ | ++ | ++ |
| | Olives | − | − | +++ | + | +++ | ++ | +++ | +++ | +++ | +++ |
| Scrapie/ | Cuneate | − | − | +/− | +/− | − | + | +/− | +/− | + | + |
| Suffolk | XII | − | − | − | +/− | +/− | − | +/− | +/− | + | + |
| | DMNV | − | − | +/− | + | + | +/− | + | + | + | + |
| | Olives | − | − | ++ | +++ | ++ | + | + | + | + | +++ |

− to ++++, increasing intensity of immunoreaction associated with intra-neuronal PrP accumulation.
− XII = hypoglossal nuclei,
DMNV = dorsal (or parasympathetic) motor nucleus of the vagal nerve;
Cuneate = lateral and accessory cuneate nuclei,
Olives = superior and inferior olivary nuclei.

Although not described in the present study the PrP labelling patterns of SSBP/1 were also compared with those of BSE agent infected sheep. In addition to differences at the obex, it is 15 likely that the variation in BSE immunolabelling is greater at more rostral brain sites. Further studies are likely to provide even more distinct differences in BSE agent associated patterns of immunolabelling in diencephalon.

Shaded areas indicate antibody panel favoured for discriminating between BSE agent infection and natural sheep scrapie.

### EXAMPLE 2

### Immunohistolochemical Differences between Natural Scrapie and the SSBP/1 Strain in Infected Sheep

A total of 43 sheep showing clinical signs consistent with scrapie were examined for PrP^{d} accumulation in the brain as described below. They were grouped according to their breed, PrP genotype and source of infection as indicated in Table 5, which also contains details on the ages or time post-inoculation at which the animals developed clinical signs. PrP genotyping was performed by sequencing using an ABI Prism 377 DNA sequencer according to the manufacturer's instructions (PE Applied Biosystems, Warrington, UK).

Experimentally infected sheep (groups 1, 2, 3 and 4) comprised four Poll-Dorset and 13 Cheviot sheep (of three different genotypes) from known scrapie-free sources, which were inoculated subcutaneously with the SSBP/1 source of scrapie as described elsewhere (Goldmann et al., 1994). Of the naturally infected animals, the seven Welsh Mountain sheep originated from a single flock in Wales and the eight Suffolk sheep came from a flock in Scotland. The 11 VRQ/ARQ Shetland sheep came from 5 different flocks in Shetland (five sheep from one flock, three sheep from another and one sheep each from the remaining three flocks).

### Samples and Immunohistochemical Procedure

The brains of the above sheep were fixed, trimmed, post-fixed and embedded according to standard procedures. Immunohistochemical examinations of all sheep brains were performed at eight different neuroanatomical locations, i.e.: cerebral cortex (frontal and occipital areas scored independently and mean value annotated), corpus striatum, hippocampus, thalamus/hypothalamus, midbrain, cerebellum (sagittal at the vermis), rostral medulla oblongata and medulla oblongata at the obex. At these levels, sections 5 μm thick were cut on a microtome, mounted on treated glass slides (Superfrost Plus, Menzel-Glaser, Germany) and dried overnight at 37° C.

Tissue sections were de-waxed and hydrated according to conventional protocols and then subjected to an antigen retrieval procedure. Sections were immersed in 98% formic acid for 5 min, washed in running tap water and then immersed in 0.2% citrate buffer and autoclaved for 5 min at 121° C. Two initial blocking steps, to quench endogenous peroxidase activity (3% hydrogen peroxide for 20 min) and to remove non-specific tissue antigens (5% normal horse serum for 60 min) were performed at room temperature. Incubation with the primary antibody (R-486—see Table 1 above, diluted 1/10 000) was performed overnight at 4° C. and the rest of the immunohistochemical protocol was performed by a commercial immunoperoxidase technique (Vector-elite ABC; Vector Laboratories, Peterborough). Sections were immersed in 0.5% copper sulphate to enhance the intensity of the immunoperoxidase reaction product and finally counterstained with Mayer's haematoxylin for 2 min.

Serial sections from a positive control block (spinal cord of a scrapie sheep) were included in each immunohistochemistry run and scored according to the procedure described below to ensure consistency in the sensitivity of the protocol. Eighteen clinically healthy sheep (three Shetland: one ARQ/ARR, one ARQ/ARQ and one ARQ/AHQ, and 15 Suffolks: nine ARQ/ARQ, three ARQ/ARR and three ARR/ARR), were subjected to the same immunohistochemical examinations to provide the negative controls.

### Nomenclature, Scoring System and Statistical Analysis

Slides were blind coded for examination. Each of the twelve different types of PrP^{d} deposition observed (see results for descriptions) was quantitatively scored (values from 0 to 3) at all the eight neuroanatomical sites examined, so that 96 numerical values were obtained from each sheep. For each animal, each “PrP^{d} type” was the mean of the values obtained at the eight different sites and the “total PrP^{d}” was the mean of the 12 PrP^{d} types. The 12 PrP^{d} types were also grouped into four “PrP^{d} patterns”, the combination of which constituted the “PrP^{d} profile”. The variations in mean values of PrP^{d} type, PrP^{d} pattern and total PrP^{d} among animals of the same group provided the standard deviations.

The magnitudes of deposition of the different PrP^{d} types and patterns and of total PrP^{d} were compared between selected sheep groups by means of unpaired t tests using a statistics computer package (InStat®, GraphPad Software, Inc., San Diego, Calif.). Parametric t tests were used when the groups under comparison presented similar standard deviations (Bartlett test) and the data had Gaussian distributions (Kolmogorov and Smirnov test); otherwise, a non-parametric t test (Mann-Whitney) was used. Comparison between SSBP/1 experimentally infected sheep groups were only performed between those in which either the breed or the genotype was in common, whereas all three possible comparisons were performed between the naturally infected sheep groups. Comparisons between naturally and experimentally infected sheep groups were only performed between those of the same genotype.

### Results

### Description of the Different PrP^{d} Types

Twelve different PrP^{d} types were identified in the 43 animals studied and scored throughout the different neuroanatomical sites. None of these types was detected in the 18 negative control Shetland and Suffolk sheep. The PrP^{d} types were:

Intraneuronal: fine to coarse, sometimes confluent granular deposits of PrP^{d} scattered in the perikarya of neurones surrounding the nucleus (FIG. 6a). They were most common in the large motor neurones of the medulla, midbrain and fastigial nucleus of the cerebellum. This type was observed in all sheep groups examined, being most conspicuous in SSBP/1 infected Cheviot sheep (FIG. 8b).

Intraglial: intense granular or ovoid deposits of PrP^{d}, slightly larger than those observed in neurones, scattered in the cell body cytoplasm of glial cells often near the nucleus (FIG. 6b). This type was neuroanatomically correlated with the intraneuronal, being most conspicuous in the brainstem and in the cerebellar white matter. This type was also found in all sheep groups and was most prominent in SSBP/1 infected Cheviot sheep (FIG. 8b).

Stellate: radiating, branching PrP^{d} deposits many times centred on a visible glial-type nucleus, conferring a star-like appearance (FIG. 6c). They could be found throughout the brain, but were most evident in the molecular layer of the cerebellar cortex followed by the cerebral grey matter, their appearance being more variable in the gray matter of the brainstem. Naturally infected Suffolk sheep were the group exhibiting the most intense and widespread stellate PrP^{d}.

Subpial: loose mesh to more amorphous, multifocal or continuous PrP^{d} accumulations beneath the pia matter (FIG. 3d). It was onspicuous in the cerebral and cerebellar cortices.

Perivascular: morphologically similar to those found underneath the pia mater, but located around the blood vessels in the white matter. They were most prominent in the thalamic/hypothalamic area of the brainstem (FIG. 3e). As with the other two glia-related PrP^{d} deposits described above, they were commonest and most pronunced in ARQ/ARQ Suffolk sheep but rarely found in SSBP/1 infected sheep.

Subependymal: morphologically similar to the above, this PrP^{d} type was found in the glial layer underneath the ependymal lining of the ventricular system (FIG. 3f). These deposits were generally discontinuous and mainly seen around the lateral ventricles, rather than the third and fourth ventricle. This type was not observed in Poll Dorset sheep and was of very low magnitude and frequency in the other SSBP/1 inoculated sheep groups. In 12 sheep, fine granular PrP^{d} deposits were observed in the cytoplasm of the ependymal cells or at their apical surface. These deposits were generally subtle and occasional and were not scored.

Linear: thread-like deposits of PrP located in the neuropil. Very thin and discrete linear forms appeared inconsistently at any brain site and were not associated topographically with other prPd types. Thicker, more intense linear staining was often associated with other PrP^{d} types and localised mainly in the medulla and the thalamus (FIG. 6g). This type was generally most prominent in naturally affected animals.

Fine punctate: numerous, small granules in the neuropil, often associated with other prPd types (FIG. 6h). It was widespread throughout all the brain areas examined where it was found with a similar low intensity in all groups except for the SSBP/1 infected Poll Dorset sheep, where it was virtually absent.

Coarse particulate: similar to the previously described fine type and many times associated with it, but made up from coarser deposits of irregular shape also located in the neuropil (FIG. 6i). It was mainly found in the brainstem and seldom seen in the cerebrum and the hippocampus and it was particularly pronounced in the Suffolk sheep.

Coalescing: almost invariably associated with the previous type, it seemed to arise by the merger of coarse particulate PrP^{d} deposits to form amorphous or mesh-like masses of immunostaining (FIG. 6j). Its anatomical distribution and its frequency and magnitude in the different groups was the same as for the coarse particulate type, but it was even rarer than this last in the experimentally challenged sheep.

Perineuronal: thin deposits of PrP^{d} around individual, scattered neuronal perikarya and neurites (FIG. 6k). It was often not associated with intraneuronal staining, but rather with coarse particulate and/or coalescing types and although it was found at many different sites, it was most common in the cerebellum. Again, it was most conspicuous in Suffolk sheep.

Plaques: fibrillar, radiate, relatively large accummulations of PrP^{d}, often unequivocally distributed around blood vessels of different caliber (FIG. 6l). The intima and media of the affected vessels also appeared partially or completely loaded with more amorphous PrP^{d} deposits. Vascular PrP^{d} plaques were almost exclusively found in the VRQ/VRQ Welsh Mountain sheep (FIGS. 9a and 10b), where they appeared focally at any brain site, but were most often found in the thalamus/hypothalamus and in the cerebral and cerebellar cortices. Intramural deposits were also observed in the meninges, but perivascular plaques were not found at this location.

### Comparative Analysis of PrP^{d} Types in Different Sheep Groups

The magnitude of total PrP^{d} (all types combined) immunolabelling in the seven groups of sheep under study is given in Table 6 and in FIG. 6I and the significance of the differences is shown in Table 7. The group of naturally infected ARQ/ARQ Suffolk sheep provided the greatest and most consistent total PrP^{d} score, which was significantly different from those of Shetland (p<0.01) and Welsh Mountain (p<0.05) sheep (Tables 2 and 3) and even greater than the scores of SSBP/1 infected sheep. On the other hand, SSBP/1 infected Poll Dorset VRQ/ARQ showed the lowest total PrP^{d} score, and the only statistical comparison performed, with Cheviot sheep of the same genotype, gave significant differences (p<0.05).

The 12 PrP^{d} types identified were grouped to form four PrP^{d} patterns in order to facilitate comparisons between the sheep groups under study. The intraneuronal and intraglial PrP^{d} were grouped as “intracellular” pattern, but were also analysed separately (Table 3). The “astrocyte-related” pattern of PrP^{d} deposits included the stellate, perivascular, subependymal and subpial types, as all of them are related to glial cells processes (the last three correspond to the so-called “glia limitans”; Jeffrey et al. 1990 J. Comp. Pathology, 103, 23-35).

Based on their location and topographic association, the fine, particulate and coalescent deposits were grouped as “neuropil” PrP^{d} pattern, together with the linear and perineuronal PrP^{d} deposits, as electron microscopy studies have shown their extracellular location (Jeffrey et al. 1994, Neuroscience Letters, 174, 39-42). Finally, PrP^{d} plaques, either perivascular or intramural, were considered together as “vascular” pattern.

The magnitudes of accumulation of these PrP^{d} patterns in the different groups of sheep appear in Table 6 and in FIGS. 8b and 8c, and the significance of the differences found in the comparisons made is shown in Table 7. The consistency of the PrP^{d} profile among the individual sheep of each group is given in FIGS. 9a, 9b and 9c. Highly consistent PrP^{d} profiles were observed in the animals experimentally challenged with the SSBP/1 scrapie source, independently of their breed or genotype (FIG. 9a). More individual variation was found within groups of naturally affected sheep, particularly in the VRQ/VRQ Welsh Mountain and VRQ/ARQ Shetland sheep groups (FIGS. 9b and 9c). In this last however, a trend for a particular PrP^{d} profile was found in the flock from which five sheep were examined.

The effect of the breed was tested by comparing the two sheep groups of the same genotype and inoculated by the same route and dose with the same scrapie source, this is, the SSBP/1 infected VRQ/ARQ Poll Dorset and Cheviot sheep. Although the PrP^{d} profile of both groups was the same, with marked predominance on intracellular deposits (FIG. 8a), differences in magnitude were significant (p<0.05). These differences were maintained for both intraneuronal and intraglial PrP^{d} (FIG. 8b) and were responsible for the significant differences found in total PrP^{d} figures.

The effect of the genotype was assessed by comparing animals of the same breed infected with the same scrapie strain, that is, the three groups of experimentally infected Cheviot sheep. All the three groups had the same PrP^{d} profile with marked predominance of intracellular deposits and total absence of vascular PrP^{d} plaques (FIG. 8a). The only difference found was in the intraglial accumulation of PrP^{d} which was significantly lower (p<0.05) in the VRQ/VRQ sheep than in the VRQ/ARQ and VRQ/ARR sheep (Table 7, FIG. 8b).

When natural scrapie sources were compared with the SSBP/1 source, major differences were found. Between SSBP/1 infected VRQ/ARQ Cheviot sheep and naturally-affected Shetland sheep of the same genotype, differences were statistically significant for the intracellular (both intraneuronal and intraglial) and astrocyte-associated PrP^{d} patterns and almost significant (p=0.06) for the neuropil PrP^{d} pattern (Table 7, FIG. 8a). However, as the PrP^{d} profile was markedly different between the two groups, differences in the magnitude of total PrP^{d} accumulation were not found. In another comparison, the VRQ/VRQ Welsh Mountain sheep differed from the SSBP/1-challenged Cheviot sheep of the same genotype in their neuropil and astrocyte-associated PrP^{d} patterns (Table 7, FIG. 8a).

When the three groups of naturally-affected sheep were compared, no differences were found in the amount of intracellular PrP^{d} (either intraneuronal or intraglial). While the magnitude of vascular PrP^{d} plaques was significantly greater in the Welsh Mountain sheep than in the other two groups (p<0.001), the ARQ/ARQ Suffolk sheep showed greater amounts of neuropil (p<0.05) and astrocyte-related (p<0.01) PrP^{d} patterns than the other two groups.

TABLE 5

| Group | Breed | Infection | Genotype | No | Age | Dpi |
|---|---|---|---|---|---|---|
| 1 | Poll-Dorset | SSBP/1 | VRQ/ARQ | 4 | | 194 (188-201) |
| 2 | Cheviot | SSBP/1 | VRQ/ARQ | 4 | | 266 (241-318) |
| 3 | Cheviot | SSBP/1 | VRQ/ARR | 4 | | 259 (244-267) |
| 4 | Cheviot | SSBP/1 | VRQ/VRQ | 5 | | 150 (136-160) |
| 5 | Welsh | Natural | VRQ/VRQ | 7 | 41 (24-72) | |
| | Mountain | | | | | |
| 6 | Shetland | Natural | VRQ/ARQ | 11 | 47 (21-96) | |
| 7 | Suffolk | Natural | ARQ/ARQ | 8 | 24 (22-30) | |

Table 5: sheep used for the study. Genotypes indicate polymorphisms at codons 136, 154 and 171 for the two alleles (V = valine, R = arginine, A = alanine, Q = glutamine).
Ages are in months and expressed as mean (minimum-maximun).
Dpi: days post-inoculation, expressed as mean (minimum-maximun).

TABLE 6

| | PrP^{d} patterns | | | | |
|---|---|---|---|---|---|
| Sheep | Intra- | Astrocyte | | | Total |
| group | cellular | -ass. | Neuropil | Vascular | PrP^{d} |
| 1 | 0.82 ± 0.24 | 0.03 ± 0.02 | 0.08 ± 0.03 | 0.00 | 0.17 ± 0.06 |
| 2 | 1.79 ± 0.36 | 0.20 ± 0.13 | 0.31 ± 0.17 | 0.00 | 0.49 ± 0.16 |
| 3 | 1.82 ± 0.46 | 0.16 ± 0.15 | 0.32 ± 0.25 | 0.00 | 0.49 ± 0.23 |
| 4 | 1.39 ± 0.16 | 0.27 ± 0.15 | 0.32 ± 0.16 | 0.00 | 0.45 ± 0.14 |
| 5 | 0.92 ± 0.69 | 0.67 ± 0.36 | 0.56 ± 0.16 | 0.52 ± 0.30 | 0.65 ± 0.28 |
| 6 | 0.52 ± 0.30 | 0.78 ± 0.17 | 0.62 ± 0.28 | 0.02 ± 0.05 | 0.61 ± 0.16 |
| 7 | 0.72 ± 0.24 | 1.36 ± 0.27 | 1.04 ± 0.43 | 0.02 ± 0.07 | 1.01 ± 0.25 |

Table 6: intensity of PrP^{d} types (grouped into four categories and total) found among the different sheep groups. Results expressed as mean ± standard deviation. For identification of the sheep groups refer to Table 1, for graphical representation to FIG. 3a and for statistical analyses to Table 3.

TABLE 7

| | 1 vs 2 | 2 vs 3 | 2 vs 4 | 3 vs 4 | 2 vs 6 | 4 vs 5 | 5 vs 6 | 5 vs 7 | 6 vs 7 |
|---|---|---|---|---|---|---|---|---|---|
| Intracellular | * | ns | ns | ns | *** | ns | ns | ns | ns |
| Intraneuronal | * | ns | ns | ns | * | ns | ns | ns | ns |
| Intraglial | * | ns | * | * | *** | ns | ns | ns | ns |
| Astrocyte- | ns | ns | ns | ns | *** | * | ns | *** | *** |
| related | | | | | | | | | |
| Neuropil | ns | ns | ns | ns | 0.06 | * | ns | * | * |
| Vascular | ND | ND | ND | ND | ND | ND | *** | *** | ns |
| Total | * | ns | ns | ns | ns | ns | ns | * | ** |

Table 7: statistical analysis of differences in magnitude of the different PrP^{d} types (intraneuronal and intraglial), patterns (intracellular, astrocyte-related, neuropil and vascular) and total PrP^{d} accumulated in the brain of scrapie-affected sheep. For identification of the sheep groups refer to Table 1.
* differences at p < 0.05;
** differences at p < 0.01;
*** differences at p < 0.001;
ns no significant differences;
ND t test not performed because the mean of one or both groups was zero.

In the report by van Keulen et al. (1995) a very similar, if not identical type to the one, described herein as stellate and with the same-neuroanatomical localisation is described. However, these authors did not identify the affected cells as astrocytes, as labelling for glial fibrillar acid protein in double-immunostained sections was negative. In contrast, it appears from these results that this type reflects PrP^{d} deposits around the processes of protoplasmic astrocytes, based on morphology (branching-stellate processes) and topography (grey matter). This type therefore was grouped together with the other three glia limitans types (perivascular, sub-ependymal and sub-pial). It is also unclear whether our coalescing type is related to the “moss-like” deposits described by Ryder et al. (2001) as these seem to be centred on neurons whereas ours are localised in the neuropil, though both seem to share neuroanatomical location, at least at the obex.

### PrP^{d} Accumulation and Clinical Disease

Some studies have indicated that it is the accumulation of PrP^{d} which is the cause of disease (DeArmond and Ironside 1999). Our results suggest that not all PrP^{d} accumulation may be linked with clinical signs of scrapie, as vascular plaques, astrocyte-associated deposits and neuropil PrP^{d} accumulations were either absent or very low in some affected animals or even groups, especially in the SSBP/1 infected sheep. Intracellular PrP^{d} deposition, particularly the intraneuronal type, showed a more consistent magnitude among the different groups studied (see FIGS. 8a and 8b) and might therefore be the most significant PrP^{d} accumulation type relative to development of clinical disease.

In the group of naturally infected Shetland sheep, seven animals had intracellular PrP^{d} scores below 0.5 (FIG. 9c), three of which had intraneuronal PrP^{d} scores were below 0.45 (data not shown), corresponding with total PrP^{d} scores also below 0.45. Also, 2/7 of the Welsh Mountain sheep had intracellular PrP^{d} scores below 0.5 (FIG. 4b) and in both intraneuronal and total PrP^{d} scores were below 0.45 (data not shown). This raises the possibility that these animals with low intraneuronal and total PrP^{d} accummtlations were subclinical cases of scrapie showing signs of a different, concurrent disease. Diagnosis of scrapie under natural conditions is often difficult, especially where it is influenced or dependent on farmer observation and therefore, some of the inter-animal variations in the PrP^{d} profile and in the magnitude of PrP^{d} accumulation might reflect incosistency in diagnostic criteria. This drawback would have had a greatest effect in the Shetland sheep of this study where cases originated from more than one holding.

### Breed, Genotype and Scrapie Source Comparisons

When comparing the Poll Dorsets VRQ/ARQ (group 1) and the Cheviots of the same genotype (group 2) infected by the same route with the same experimental source of scrapie, the results show no effect of the breed of sheep in the PrP^{d} profile. There were however significant differences in the intensity of intracellular and total PrP^{d} deposition, but not in the other three patterns. The assessment of the effect of sheep genotype on PrP^{d} accumulation in the brain was achieved by comparing the SSBP/1 infected Cheviots of three different genotypes (VRQ/ARQ, VRQ/ARR and VRQ/VRQ; groups 2, 3 and 4, respectively). Minor, though significant, differences were found in the amount of intraglial PrP^{d} accumulation which was lower in the animals of the VV₁₃₆ genotype, but no differences were found in the PrP^{d} profile or in the total PrP^{d} accumulation.

What can be the effects of breed and PrP genotype if they do not affect the profile of PrP^{d} deposition in the brain? The lower amounts of PrP^{d} accumulated in the brain of the Poll Dorset sheep corresponds to a shorter incubation period in this breed than that of Cheviot sheep of the same or very similar genotype (groups 2 and 3, both VA at codon 136, see Table 1). However, the incubation period in the VRQ/VRQ Cheviot sheep was the shortest of all the SSBP/1-inoculated sheep, yet they showed greater PrP^{d} accumulation in the brain than the Poll Dorset sheep. Assuming that all sheep were killed at comparable time points during the course of the clinical disease, it is hypothesised that the PrP genotype (at least at codon 136) influences the rapidity of PrP^{d} accumulation in the brain, and hence the shorter incubation period of the VRQ/VRQ Cheviot sheep. In contrast, it is also possible that breed factors other than the PrP genotype could influence incubation period and clinical signs, so that lower PrP^{d} levels of deposition might be required to cause neurodegeneration in some breeds relative to others. This could explain the lower amount of PrP^{d} found in the short incubation Poll Dorset sheep, when compared with the same infection in longer incubation Cheviot sheep of the same genotype.

The main conclusion from the examination of the SSBP/1 infected sheep is that infection by the same strain produces a highly consistent PrP^{d} deposition profile with only slight differences in magnitude. This is perhaps surprising as SSBP/1 is a pool of sheep scrapie brains, from which different murine isolates, most notably 22A, 79A and 139A, have been identified through passages in sheep and cloning in mice (Dickinson, 1976). The consistency of PrP^{d} patterns obtained in SSBP/1 sheep brains might be because 1) multiple strains exist as a stable equilibrium in the SSBP/1 pool, or 2) because a single strain within the pool exerts a dominant pathogenetic effect. Alternatively, and perhaps most plausibly, the SSBP/1 derived murine isolates may be generated following mutation or interspecies passage.

The effect of the scrapie source on the PrP^{d} profile becomes more evident when naturally affected sheep are compared. The consistent detection of cerebrovascular PrP^{d} plaques in the Welsh Mountain sheep clearly cannot merely reflect PrP genotype differences, as they were of the same genotype as the SSBP/1 infected VRQ/VRQ Cheviot sheep where plaques were absent. These plaques are also unlikely to be a breed effect, as no breed effect on PrP^{d} profile was found when the SSBP/1 infected Poll Dorset sheep were compared to SSBP/1 infected Cheviot sheep. Moreover, previous studies on natural sheep scrapie did not find any association between the breed and the presence of cerebrovascular amyloid plaques in the brain (Gilmour et al. 1985). It appears therefore that this PrP^{d} type/pattern reflects the properties of a particular scrapie strain(s), a notion that is in agreement with findings in murine scrapie (Bruce et al. 1976, 1989).

The PrP^{d} profile found in naturally infected ARQ/ARQ Suffolk sheep, with a consistent predominance of astrocyte-associated or neuropil PrP^{d} and almost complete lack of vascular plaques, was different from that in naturally infected Welsh Mountain sheep. On their own, these differences are difficult to interpret as many factors can be taken into account. However, if the breed and genotype effects are ruled out from the comparisons already described, it seems probable that differences in the PrP^{d} profile are mainly driven by properties of the scrapie strain.

The group of naturally infected VRQ/ARQ Shetland sheep contained the most heterogeneous individuals of all, something that could be explained by the diversity of flocks of origin. As a group, its PrP^{d} profile was notably different from that of SSBP/1 infected sheep and of Welsh Mountain sheep. Following the reasoning described above, these differences probably derive from strain diversity. Although its PrP^{d} profile was very similar to that of Suffolk sheep, the magnitude of PrP^{d} deposition (astrocyte-related, neuropil and total) was lower, coinciding with a higher mean age of outcome of disease (47 vs 24 months, see Table 1). Assuming a similar age at infection of both groups, it would appear that, in line with the hypothesis expressed for the experimentally infected groups, the genotype (VRQ/ARQ vs ARQ/ARQ, in this case) influences the rapidity of accumulation of PrP^{d} in the brain, and thus the incubation period.

In conclusion, this Example confirms that the profile of PrP^{d} deposition in the brain, defined as the combination of different PrP^{d} patterns and types, is characteristic of scrapie strains in sheep. As such, it suggests that the immunohistological technique reported in Example 1 will be effective to distinguish between a range of TSE strains.

1. A method for typing a strain of a transmissible spongiform encephalopathy (TSE) in an infected animal, said method comprising detecting the presence or concentration of a peptide sequence within a cell of a particular type taken from said animal, wherein the presence or concentration of said peptide sequence within said cell type is characteristic of a particular strain of TSE.

2. A method according to claim 1 wherein said particular cell type is a brain cell, a central nervous system (CNS) cell, or cell of lymphoid tissues.

3. A method according to claim 1 of claim 2 wherein the presence of said peptide sequence within a particular cell type is characteristic of a particular strain of TSE.

4. A method according to claim 3 wherein the animal is a sheep.

5. A method according to claim 3 or claim 4 wherein the peptide sequence binds to an antibody raised to a peptide corresponding to amino acids 84-105 of the prion protein of ovine spongiform encephalopathy or an epitopic region thereof, and its presence in a glial cell is detected to distinguish between scrapie of bovine spongiform encephalopathy (BSE) derived strains.

6. A method according to claim 1 or claim 2 wherein the concentration of said peptide sequence within a particular cell type is characteristic of a particular strain of TSE, and the result from a test animal is compared with results obtained in a similar manner from cells of at least one comparative animal suffering from a known strain of TSE.

7. A method according to claim 6 wherein the said cells are derived from a sheep.

8. A method according to claim 7, wherein the said peptide sequence binds to an antibody raised to a peptide corresponding to amino acids 84-105 of the prion protein of ovine spongiform enpephalopathy or an epitopic region thereof, its concentration in neuronal cells is detected, and the said known strain of TSE is scrapie and/or a bovine spongiform encephalopathy (BSE) derived strain.

9. A method according to claim 8 wherein the peptide sequence detected binds to an antibody raised to a peptide corresponding to amino acids 89-104 of ovine spongiform encephalopathy or an epitopic region thereof.

10. A method according to claim 7, wherein the said peptide sequence binds to an antibody raised to a peptide corresponding to amino acids 217-231 of the prion protein of bovine spongiform encephalopathy or an epitopic region thereof, and its concentration in neuronal cells is detected, and the said known strain of TSE is scrapie and/or a bovine spongiform encephalopathy (BSE) derived strain.

11. A method according to claim 7 wherein the peptide sequence binds to an antibody raised to a peptide corresponding to amino acids 54-60 of sheep prion protein or an epitopic region thereof, and its concentration in extracellular neuropil is detected, and the said known strain of TSE is scrapie and/or a bovine spongiform encephalopathy (BSE) derived strain.

12. A method according to any one of the preceding claims wherein the presence or concentration of said peptide sequence is determined using an antibody or specific binding fragment thereof, which is specific for said peptide sequence.

13. A method according to claim 12 wherein the detection is effected by means of an ELISA assay.

14. A method according to any one of the preceding claims which is carried out as a cellular assay.

15. A method according to any one of claims 1 to 3 wherein said particular cell types cells are separated prior to detection of the peptide sequence.

16. A method according to claim 15 wherein the cells are separated by flow cytometry.

17. A method according to claim 15 or claim 16 wherein said cells are lysed prior to detection of the presence or amount of peptide sequence.

18. A kit for typing a strain of a transmissible spongiform encephalopathy (TSE) using a method according to any one of the preceding claims, said kit comprising an antibody or a binding fragment thereof which is specific for a peptide sequence derived from a TSE which is found within a cell of a particular cell type of an infected animal and wherein the presence or concentration of said peptide sequence within said cell type is characteristic of a particular strain of TSE, and means for detecting said antibody.

19. A method for typing a strain of a transmissible spongiform encephalopathy (TSE) in an infected animal substantially as hereinbefore described.


SEQUENCE LISTING (20 sequences)

SEQ ID NO: 1 - protein, 256 aa, Ovis aries
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGGWGQGGSHSQWNKPSKPKTNMKHVAGAAAAGAVVGGLGGYMLGSAMSRPLIHFGNDYEDRYYRENMYRYPNQVYYRPVDQYSNQNNFVHDCVNITVKQHTVTTTTKGENFTETDIKIMERVVEQMCITQYQRESQAYYQRGASVILFSSPPVILLISFLIFLIVG

SEQ ID NO: 2 - protein, 264 aa, Bos taurus
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGWGQPHGGGGWGQGGTHGQWNKPSKPKTNMKHVAGAAAAGAVVGGLGGYMLGSAMSRPLIHFGSDYEDRYYRENMHRYPNQVYYRPVDQYSNQNNFVHDCVNITVKEHTVTTTTKGENFTETDIKMMERVVEQMCITQYQRESQAYYQRGASVILFSSPPVILLISFLIFLIVG

SEQ ID NO: 3 - protein, 256 aa, Ovis aries
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGGWGQGGTHGQWNKPSKPKTNMKHVAGAAAAGAVVGGLGGYMLGSAMSRPLIHFGSDYEDRYYRENMHRYPNQVYYRPVDQYSNQNNFVHDCVNITVKEHTVTTTTKGENFTETDIKMMERVVEQMCITQYQRESQAYYQRGASVILFSSPPVILLISFLIFLIVG

SEQ ID NO: 4 - protein, 256 aa, Ovis aries
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGGWGQGGSHSQGNKPSKPKTNMKHVAGAAAAGAVVGGLGGYMLGSAMSRPLIHFGNDYEDRYYRENMYRYPNQVYYRPVDQYSNQNNFVHDCVNITVKQHTVTTTTKGENFTETDIKIMERVVEQMCITQYQRESQAYYQRGASVILFSSPPVILLISFLIFLIVG

SEQ ID NO: 5 - protein, 256 aa, Ovis aries
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGGWGQGGSHSQWNKPSKPKTNTKHVAGAAAAGAVVGGLGGYMLGSAMSRPLIHFGNDYEDRYYRENMYRYPNQVYYRPVDQYSNQNNFVHDCVNITVKQHTVTTTTKGENFTETDIKIMERVVEQMCITQYQRESQAYYQRGASVILFSSPPVILLISFLIFLIVG

SEQ ID NO: 6 - protein, 256 aa, Ovis aries
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGGWGQGGSHSQWNKPSKPKTNMKHVAGAAAAGAVVGGLGGYMLGSVMSRPLIHFGNDYEDRYYRENMYRYPNQVYYRPVDQYSNQNNFVHDCVNITVKQHTVTTTTKGENFTETDIKIMERVVEQMCITQYQRESQAYYQRGASVILFSSPPVILLISFLIFLIVG

SEQ ID NO: 7 - protein, 256 aa, Ovis aries
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGGWGQGGSHSQWNKPSKPKTNMKHVAGAAAAGAVVGGLGGYMLGSTMSRPLIHFGNDYEDRYYRENMYRYPNQVYYRPVDQYSNQNNFVHDCVNITVKQHTVTTTTKGENFTETDIKIMERVVEQMCITQYQRESQAYYQRGASVILFSSPPVILLISFLIFLIVG

SEQ ID NO: 8 - protein, 256 aa, Ovis aries
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGGWGQGGSHSQWNKPSKPKTNMKHVAGAAAAGAVVGGLGGYMLGSATSRPLIHFGNDYEDRYYRENMYRYPNQVYYRPVDQYSNQNNFVHDCVNITVKQHTVTTTTKGENFTETDIKIMERVVEQMCITQYQRESQAYYQRGASVILFSSPPVILLISFLIFLIVG

SEQ ID NO: 9 - protein, 256 aa, Ovis aries
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGGWGQGGSHSQWNKPSKPKTNMKHVAGAAAAGAVVGGLGGYMLGSAMNRPLIHFGNDYEDRYYRENMYRYPNQVYYRPVDQYSNQNNFVHDCVNITVKQHTVTTTTKGENFTETDIKIMERVVEQMCITQYQRESQAYYQRGASVILFSSPPVILLISFLIFLIVG

SEQ ID NO: 10 - protein, 256 aa, Ovis aries
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGGWGQGGSHSQWNKPSKPKTNMKHVAGAAAAGAVVGGLGGYMLGSAMSRPFIHFGNDYEDRYYRENMYRYPNQVYYRPVDQYSNQNNFVHDCVNITVKQHTVTTTTKGENFTETDIKIMERVVEQMCITQYQRESQAYYQRGASVILFSSPPVILLISFLIFLIVG

SEQ ID NO: 11 - protein, 256 aa, Ovis aries
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGGWGQGGSHSQWNKPSKPKTNMKHVAGAAAAGAVVGGLGGYMLGSAMSRPLMHFGNDYEDRYYRENMYRYPNQVYYRPVDQYSNQNNFVHDCVNITVKQHTVTTTTKGENFTETDIKIMERVVEQMCITQYQRESQAYYQRGASVILFSSPPVILLISFLIFLIVG

SEQ ID NO: 12 - protein, 256 aa, Ovis aries
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGGWGQGGSHSQWNKPSKPKTNMKHVAGAAAAGAVVGGLGGYMLGSAMSRPLIRFGNDYEDRYYRENMYRYPNQVYYRPVDQYSNQNNFVHDCVNITVKQHTVTTTTKGENFTETDIKIMERVVEQMCITQYQRESQAYYQRGASVILFSSPPVILLISFLIFLIVG

SEQ ID NO: 13 - protein, 256 aa, Ovis aries
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGGWGQGGSHSQWNKPSKPKTNMKHVAGAAAAGAVVGGLGGYMLGSAMSRPLIHFGNDYEDCYYRENMYRYPNQVYYRPVDQYSNQNNFVHDCVNITVKQHTVTTTTKGENFTETDIKIMERVVEQMCITQYQRESQAYYQRGASVILFSSPPVILLISFLIFLIVG

SEQ ID NO: 14 - protein, 256 aa, Ovis aries
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGGWGQGGSHSQWNKPSKPKTNMKHVAGAAAAGAVVGGLGGYMLGSAMSRPLIHFGNDYEDRYYHENMYRYPNQVYYRPVDQYSNQNNFVHDCVNITVKQHTVTTTTKGENFTETDIKIMERVVEQMCITQYQRESQAYYQRGASVILFSSPPVILLISFLIFLIVG

SEQ ID NO: 15 - protein, 256 aa, Ovis aries
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGGWGQGGSHSQWNKPSKPKTNMKHVAGAAAAGAVVGGLGGYMLGSAMSRPLIHFGNDYEDRYYRENMYRYPNQVYYRPVDRYSNQNNFVHDCVNITVKQHTVTTTTKGENFTETDIKIMERVVEQMCITQYQRESQAYYQRGASVILFSSPPVILLISFLIFLIVG

SEQ ID NO: 16 - protein, 256 aa, Ovis aries
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGGWGQGGSHSQWNKPSKPKTNMKHVAGAAAAGAVVGGLGGYMLGSAMSRPLIHFGNDYEDRYYRENMYRYPNQVYYRPVDHYSNQNNFVHDCVNITVKQHTVTTTTKGENFTETDIKIMERVVEQMCITQYQRESQAYYQRGASVILFSSPPVILLISFLIFLIVG

SEQ ID NO: 17 - protein, 256 aa, Ovis aries
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGGWGQGGSHSQWNKPSKPKTNMKHVAGAAAAGAVVGGLGGYMLGSAMSRPLIHFGNDYEDRYYRENMYRYPNQVYYRPVDKYSNQNNFVHDCVNITVKQHTVTTTTKGENFTETDIKIMERVVEQMCITQYQRESQAYYQRGASVILFSSPPVILLISFLIFLIVG

SEQ ID NO: 18 - protein, 256 aa, Ovis aries
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGGWGQGGSHSQWNKPSKPKTNMKHVAGAAAAGAVVGGLGGYMLGSAMSRPLIHFGNDYEDRYYRENMYRYPNQVYYRPVDQYSNQNNFVHDCVNITVKQHTVTTTTKGENFTETDIKIMEQVVEQMCITQYQRESQAYYQRGASVILFSSPPVILLISFLIFLIVG

SEQ ID NO: 19 - protein, 256 aa, Ovis aries
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGGWGQGGSHSQWNKPSKPKTNMKHVAGAAAAGAVVGGLGGYMLGSAMSRPLIHFGNDYEDRYYRENMYRYPNQVYYRPVDQYSNQNNFVHDCVNITVKQHTVTTTTKGENFTETDIKIMERVVEQMCITQYQRESQAYYQRGASVILFSPPPVILLISFLIFLIVG

SEQ ID NO: 20 - protein, 256 aa, Ovis aries
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGGWGQGGSHSQWNKPSKPKTNMKHVAGAAAAGAVVGGLGGYMLGSAMSRPLIHFGNDYEDRYYRENMYRYPNQVYYRPVDQYSNQNNFVHDCVNITVKQHTVTTTTKGENFTETDIKIMERVVEQMCITQYQRESQAYYQRGASVILFSSSPVILLISFLIFLIVG